SEQUENCE LISTING (3 sequences)

SEQ ID NO: 1 - DNA, 21 bp, Artificial Sequence
  oligonucleotide corresponding to 3.5 repetitions of the human telomeric motif (G-rich strand)
gggttagggttagggttaggg

SEQ ID NO: 2 - DNA, 18 bp, Artificial Sequence
  oligonucleotide TS for extension reaction and PCR amplification of extension products in telomerase assay
aatcgttcgagcagagtt

SEQ ID NO: 3 - DNA, 27 bp, Artificial Sequence
  oligonucleotide CXext for PCR amplification of extension products in telomerase assay
gtgcccttacccttacccttaccctaa

(19) United States
(12) Patent Application Publication
(10) Pub. No.: US 2003/0013711 A1
(43) Pub. Date: Jan. 16, 2003

(54) Chemical derivatives and their application as antitelomerase agents

(76) Inventors: Patrick Mailliet, Fontenay Sous Bois (FR); Jean-Francois Riou, Reims (FR); Abdelazize Laoui, Bridgewater, NJ (US); Jean-Louis Mergny, Villejuif (FR); Sylvie Gontier, Saint Ouen L'aumone (FR)

Correspondence Address: Finnegan, Henderson, Farabow,, Garrett & Dunner, L.L.P., 1300 I Street, N.W., Washington, DC, 20005-3315, US

(21) Appl. No.: 10/079,500

(22) Filed: Feb. 22, 2002

Related U.S. Application Data
(60) Provisional application No. 60/282,862, filed on Apr. 11, 2001.

(30) Foreign Application Priority Data
Feb. 23, 2001 (FR) 0102461

(51) Int. Cl. A61K 31/53; C 07D 4 3/02

(52) U.S. Cl. 514/241; 544/198; 544/212

(57) ABSTRACT

The present invention relates to cancer therapies using novel anticancer agents with a specific mechanism of action. The invention also relates to novel chemical compounds and their therapeutic application in man.

This application claims the benefit of priority from French Application No. 0102461, filed Feb. 23, 2001, and U.S. Provisional Application No. 60/282,862, filed Apr. 11, 2001, which are both incorporated herein by reference in their entirety.

The present invention relates to cancer therapy and novel anticancer agents having a very specific mechanism of action. The invention also relates to novel chemical compounds and their therapeutic application in man.

The present invention relates to the use of novel non-nucleotide chemical compounds which interact with specific deoxyribonucleic acid (DNA) or ribonucleic acid (RNA) structures. These novel compounds generally contain a distributor agent linked to an amino aromatic group. These novel compounds are useful in the treatment of cancers and typically act as telomerase inhibitors. They are also useful for stabilizing DNA in G-quadruplex structures (guanine tetrads). The inhibition of telomerase via the stabilization of these G-quadruplexes generally results in the termination of cellular mitosis and the death of rapidly-dividing cells such as cancer cells. It may also result in the induction of senescence in cancer cells. Thus, such telomerase inhibiting agents have important therapeutic applications.

The compounds of the present invention have the advantage from the therapeutic viewpoint of blocking telomerase. From the biological viewpoint, telomerase allows the addition of repeated DNA sequences of the type TTAGGG, which are known as telomeric sequences, to the end of the telomere during cell division. By this action, telomerase makes the cell immortal. Specifically, in the absence of this enzymatic activity, the cell loses 100 to 150 bases at each division, which rapidly renders it senescent. During the development of rapidly dividing cancer cells, these cells have telomeres that are maintained at a stable length throughout the cell division. In these cancer cells, telomerase is highly activated and allows the addition of repeated units of telomeric sequences to the end of the telomeres. Thus, the length of the telomere in the cancer cells is conserved. It has recently been found that more than 85% of cancer cells test positive for the presence of telomerase, whereas somatic cells do not have this characteristic.

Thus, telomerase is an important target for treating cancer cells. The first approach for blocking telomerase involved the use of nucleotide structures (Chen et al., Proc. Natl. Acad. Sci. USA 93(7), 2635-2639). Diaminoanthraquinones (Sun et al., J. Med. Chem. 40(14), 2113-6) and diethyloxadicarbocyanins (Wheelhouse R. T. et al., J. Am. Chem. Soc. 120:3261-2, 1998) are among the non-nucleotide compounds which have been used.

Patent WO 99/40087 describes the use of compounds which interact with the G-quadruplex structures. Such G-quadruplex structures are typically perylene compounds and carbocyanins containing at least seven rings, including two heterocycles.

It has been found, entirely surprisingly, that the simple structures of the present invention can obtain at least an equivalent result compared to structures that are much more chemically complicated (such as diaminoanthraquinones, diethyloxadicarbocyanins, perylene compounds and carbocyanins containing at least seven rings). The compounds of the present invention which satisfy the intended objective, that is to say which bind the G-quadruplex structure of DNA or of RNA, such as the G-quadruplex structure of telomeres, and have inhibitory activity on telomerases, correspond to the following general formula:

nitrogenous aromatic ring —NR₃— distributor —O— or —S— non-aromatic hydrocarbon-based chain or nitrogenous aromatic ring

in which

the nitrogenous aromatic ring represents:

a quinoline optionally substituted with at least

one group N(Ra)(Rb) in which Ra and Rb, which may be identical or different, represent hydrogen or a C1-C4 alkyl radical, or

a group ORa in which Ra is defined as above

a quinoline containing a nitrogen atom in quaternary form, or

a benzamidine, or

a pyridine;

the distributor represents:

a triazine group optionally substituted with an alkyl radical containing 1 to 4 carbon atoms, a thio, oxy or amino radical which are themselves optionally substituted with one or more short-chain alkyl chains containing 1 to 4 carbon atoms or alternatively a halogen atom, or

a carbonyl group, or

a C(═NH)—NH—C(═NH) group, or

an alkyldiyl group containing 3 to 7 carbon atoms, or

a diazine group optionally substituted with the same groups as the triazine;

R₃ presents hydrogen or a C1-C4 alkyl radical;

or a salt thereof.

Within the meaning of the above formula, the expression “non-aromatic hydrocarbon-based chain” means a linear or branched alkyl (C1-C4) or alkenyl (C2-C4) chain or a cycloalkyl (C3-8), cycloalkenyl (C3-C18) or heterocycloalkyl (C3-C18) chain. The heterocycloalkyl group optionally includes the nitrogen atom of an amino, alkylamino, arylamino, dialkyl amino, diarylamino, or combinations thereof.

It is understood that the non-aromatic hydrocarbon-based chain may be optionally substituted with one or more atoms or radicals chosen from halogen, hydroxyl, aryl, heteroaryl, alkyloxy, aryloxy, thio, alkylthio, arylthio, amino, alkylamino, arylamino, dialkylamino, diarylamino, amidino, guanidino, alkylcarbonylamino, arylcarbonylamino, carboxyl, alkyloxycarbonyl, aryloxycarbonyl, aminocarbonyl, alkylaminocarbonyl, arylaminocarbonyl, dialkylaminocarbonyl, alkylcarbonyl, arylcarbonyl, cyano, trifluoromethyl, and combinations thereof.

The alkyl chains of the optional substituent of the hydrocarbon-based chain may contain 1 to 4 carbon atoms; and the aryl groups of the optional substituents of the hydrocarbon-based chain may contain 5 to 18 carbon atoms.

In one embodiment, the compounds include a distributor comprising a triazine or diazine group. Suitable diazine groups include pyrimidines and quinazolines. The hydrocarbon-based chains may be alkyl chains containing 2 or 3 carbon atoms; and heterocycloalkyl or cycloalkyl chains may contain 5 to 7 carbon atoms or, alternatively, 4 to 7 carbon atoms.

Suitable triazines include the compounds corresponding to formula (I) below:

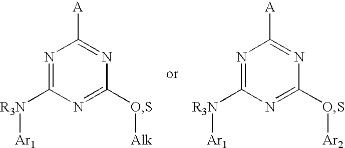

in which:

A represents

an amino group of formula NR₁R₂ in which R₁ and R₂, which may be identical or different, represent hydrogen or a straight or branched alkyl group containing 1 to 4 carbon atoms, or

a group OR₁ or SR₁ in which R₁ has the same meaning as above, or

an alkyl group containing 1 to 4 carbon atoms or a trifluoromethyl group, or

a hydrogen atom, or

a halogen atom chosen from fluorine, chlorine, bromine and iodine;

—R₃ represents hydrogen or a C1-C4 alkyl radical;

Ar₁ and Ar₂, which may be identical or different, represent:

a quinoline optionally substituted with

at least one group N(Ra)(Rb) in which Ra and Rb, which may be identical or different, represent hydrogen or a C1-C4 alkyl radical, or

a group ORa in which Ra is defined as above

a quinoline containing a nitrogen atom in quaternary form, or

a benzamidine, or

a pyridine attached at the 4-position or fused with an aryl or heteroaryl group optionally substituted with a C1-C4 alkyl group;

Alk represents an optionally substituted non-aromatic hydrocarbon-based chain chosen from linear or branched alkyl (C1-C4), linear or branched alkenyl (C2-C4), cycloalkyl (C3-C18), cycloalkenyl (C3-C18), and heterocycloalkyl (C3-C18) chains. The heterocycloalkyl may optionally include the nitrogen atom of an amino, alkylamino, arylamino, dialkylamino, diarylamino, or combination thereof;

or a salt thereof.

In the compounds defined above, Alk may represent

a linear or branched alkyl unit containing 1 to 4 carbon atoms, optionally substituted with an amino, alkylamino, arylamino, dialkylamino, diarylamino, or combination thereof

a linear or branched alkenyl unit containing 2 to 4 carbon atoms, optionally substituted with an amino, alkylamino, arylamino, dialkylamino, diarylamino, or combination thereof

a cycloalkyl unit containing from 3 to 18 carbon atoms

a cycloalkenyl unit containing from 3 to 18 carbon atoms

a heterocycloalkyl unit containing from 3 to 18 carbon atoms, optionally including the nitrogen atom of an amino, alkylamino, arylamino, dialkylamino, diarylamino, or combination thereof;

or a salt thereof.

In another embodiment, Alk represents

a linear or branched alkyl unit containing 2 or 3 carbon atoms, substituted with an amino, alkylamino, arylamino, dialkylamino, diarylamino, or combination thereof

an alkenyl unit containing 2 or 3 carbon atoms, substituted with an amino, alkylamino, arylamino, dialkylamino, diarylamino, or combination thereof

a heterocycloalkyl unit containing from 4 to 7 carbon atoms and including a nitrogen atom of an amino, alkylamino, arylamino, dialkylamino, diarylamino, or combination thereof;

a salt thereof.

A suitable heterocycloalkyl unit, for example, is the piperidyl radical.

It is obvious that the quinoline units may be substituted with any other group not involved in the intended application; thus, acridine, isoquinoline, quinazoline, quinoxaline, phthalazine, benzothiazine, benzoxazine, phenoxazine, and phenothiazine groups are included in the definition of the quinoline groups.

The compounds of formula (I) include those for which Ar₁ and/or Ar₂ is chosen from 4-aminoquinolyl, 4-alkyl- or 4-dialkylamino-quinolyl, 4-aminoquinolinium or quinolinium groups in which the quinolinium ring is optionally substituted with a methyl group.

Group A may represent a methylthio, amino, alkylamino or dialkylamino radical, in which the radicals in the alkyl groups contain 1 to 4 carbon atoms.

The non-aromatic hydrocarbon-based chain Alk may represent a 2-(dialkylamino)ethyl, 3-(dialkylamino)propyl, 2-(N-alkyl-N-arylamino)ethyl, 3-(N-alkyl-N-arylamino)propyl, N-alkylpiperidyl or N-arylpiperidyl chain in which the alkyl groups contain 1 to 4 carbon atoms, or in an alternative embodiment, 1 or 2 carbon atoms; and the aryl groups contain 5 to 18 carbon atoms, or in an alternative embodiment, 6 carbon atoms.

Another subject of the present invention relates to the compounds of formula (I) as novel chemical products. The subject thus relates to the novel products characterized in that they correspond to the following general formula:

nitrogenous aromatic ring —NR₃— distributor —O— or —S— non-aromatic hydrocarbon-based chain or nitrogenous aromatic ring

in which

the nitrogenous aromatic ring represents:

a quinoline optionally substituted with at least

one group N(Ra)(Rb) in which Ra and Rb, which may be identical or different, represent hydrogen or a C1-C4 alkyl radical, or

a group ORa in which Ra is defined as above

a quinoline containing a nitrogen atom in quaternary form, or

a benzamidine, or

a pyridine;

R₃ represents hydrogen or a C1-C4 alkyl radical;

the distributor represents:

a triazine group optionally substituted with an alkyl radical containing 1 to 4 carbon atoms, a thio, oxy or amino radical which are themselves optionally substituted with one or more short-chain alkyl chains containing 1 to 4 carbon atoms or alternatively a halogen atom, or

a diazine group optionally substituted with the same groups as the triazine;

the non-aromatic hydrocarbon-based chain is chosen from linear or branched alkyl (C1-C4), linear or branched alkenyl (C2-C4), cycloalkyl (C3-C18), cycloalkenyl (C3-C18), and heterocycloalkyl (C3-C18) chains. The heterocycloalkyl may optionally include the nitrogen atom of an amino, alkylamino, arylamino, dialkylamino, diarylamino, or combination thereof;

or a salt thereof.

The present invention also relates to the novel products corresponding to formula (I) below:

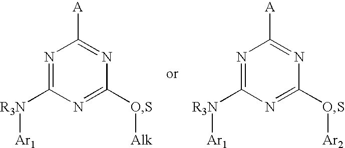

in which:

A represents

an amino group of formula NR₁R₂ in which R₁ and R₂, which may be identical or different, represent a hydrogen atom or a straight or branched alkyl group containing 1 to 4 carbon atoms, or

a group OR₁ or SR₁ in which R₁ represents hydrogen or has the same meaning as above, or

an alkyl group containing 1 to 4 carbon atoms or a trifluoromethyl group, or a hydrogen atom, or

a halogen atom chosen from fluorine, chlorine, bromine and iodine;

R₃ represents a hydrogen atom or a C1-C4 alkyl group;

Ar₁ and Ar₂, which may be identical or different, represent:

a quinoline optionally substituted with at least

one group N(Ra)(Rb) in which Ra and Rb, which may be identical or different, represent hydrogen or a C1-C4 alkyl radical or

a group ORa in which Ra is defined as above,

a quinoline containing a nitrogen atom in quaternary form, or

a benzamidine, or

a pyridine attached in position −4 or fused with an aryl or heteroaryl group optionally substituted with a C1-C4 alkyl group;

Alk represents an optionally substituted non-aromatic hydrocarbon-based chain chosen from linear or branched alkyl (C1-C4), linear or branched alkenyl (C2-C4), cycloalkyl (C3-C18), cycloalkenyl (C3-C18), and heterocycloalkyl (C3-C18) chains. The heterocycloalkyl optionally includes the nitrogen atom of an amino, alkylamino, arylamino, dialkylamino, diarylamino, or combination thereof;

or a salt thereof.

In the above novel compounds, Alk may represent a group already indicated above. In one embodiment, Alk represents

a linear or branched alkyl unit containing 2 or 3 carbon atoms, substituted with an amino, alkylamino, arylamino, dialkylamino, diarylamino, or combination thereof

an alkenyl unit containing 2 or 3 carbon atoms, substituted with an amino, alkylamino, arylamino, dialkylamino, diarylamino, or combination thereof

a heterocycloalkyl unit containing from 5 to 7 carbon atoms including the nitrogen atom of an amino, alkylamino, arylamino, dialkylamino, diarylamino, or combination thereof;

or a salt thereof.

Suitable Ar₁ groups include, but are not limited to: 4-amino- or 4-methylamino- or 4-dimethylamino-quinolyl or quinolinium in which the quinolinium ring is optionally substituted with a methyl group.

Suitable A groups include, but are not limited to, an amino, dimethylamino, or methylthio group.

Suitable Alk groups include, but are not limited to, alkyl chains containing 2 or 3 carbon atoms, cycloalkyl or heterocycloalkyl chains containing 5 to 7 carbon atoms, wherein these chains themselves contain an amino, alkylamino, arylamino, dialkylamino, diarylamino, or combination thereof, in which the alkyl groups contain 1 to 4 carbon atoms and the aryl groups contain 5 to 18 carbon atoms.

Examples of non-aromatic hydrocarbon-based chain Alk groups include 2-(dialkylamino)ethyl, 3-(dialkylamino)propyl, 2-(N-alkyl-N-arylamino)ethyl, 3-(N-alkyl-N-arylamino)propyl, N-alkylpiperidyl and N-arylpiperidyl chain. The alkyl groups may contain 1 to 4 carbon atoms, or in an alternative embodiment, 1 or 2 carbon atoms; and the aryl groups may contain 5 to 18 carbon atoms, or in an alternative embodiment, 6 carbon atoms.

Exemplary compounds having the above described formula include:

N6-[6-(2-dimethylaminoethoxy)-4-methylsulfanyl-[1,3,5]triazin-2-yl]-2-methylquinoline-4,6-diamine,

N6-[6-(3-dimethylaminopropoxy)-4-methylsulfanyl-[1,3,5]triazin-2-yl]-2-methylquinoline-4,6-diamine,

N6-[6-(1-methylpiperidin-4-yloxy)-4-methylsulfanyl-[1,3,5]triazin-2-yl]-2-methylquinoline-4,6-diamine, and

N6-[6-(2-dimethylaminoethylsulfanyl)-4-methylsulfanyl-[1,3,5]triazin-2-yl]-2-methylquinoline-4,6-diamine.

Another subject of the present invention relates to the use of the compounds of formula (I) as pharmaceutical products for human use.

General Method 1

The processes for preparing the compounds of formula (I)

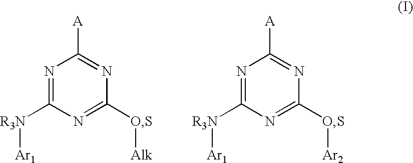

are described below.

When Ar₁ and Alk are present, the triazine of general formula (A) may be obtained by sequential displacement of the halogen atoms, very generally of the chlorine atoms, from the products of general formula (B) by the amines Ar₁NHR₃ of general formula (C), and then with alcohols or phenols or thiols or thiophenols of general formula (E) or (E′), according to Scheme 1. Alternatively, (B) may be reacted with (E) or (E′), and then the intermediate obtained with (C), according to Scheme 1:

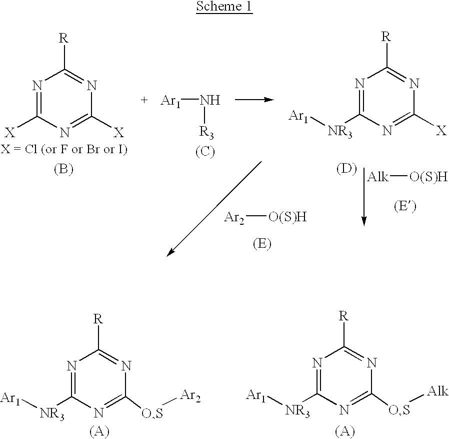

In the compounds (B), (D) and (A) of Scheme 1, R represents the values of A as defined above in the compounds of formula (I). If necessary, the potentially reactive functions of A may be optionally protected according to the usual methods known to those skilled in the art.

Ar₁, Ar₂, Alk and R₃ have the meanings given above for the compounds of formula (I).

In general, to prepare the intermediate D, the process is performed with 1 mol of dihalo-s-triazine or trihalo-s-triazine, and 1 mol of amine Ar₁. The process may be performed in an inert solvent, such as acetone, which is optionally aqueous; or an alcohol which is optionally aqueous, for instance ethanol; or a halogenated solvent, such as dichloromethane; or an ether, such as diethyl ether or dioxane; or a polar aprotic solvent, such as DMF, DMSO or NMP. In one embodiment, the process is performed at a temperature of between 20° C. and 50° C.

The product of general formula (D) is advantageously isolated and purified.

One mol of alcohol Alk-OH, thiol Alk-SH, phenol Ar₂—OH, or thiophenol Ar₂—SH, is then added to the product of general formula (D), which may optionally be isolated. The process is typically performed at a temperature of between 50° C. and the dry reflux temperature, or in a solvent, for instance an ether such as dioxane, or a polar aprotic solvent such as DMF, DMSO or NMP. The process is advantageously performed by reacting the corresponding alkoxides Alk-ONa, thioalkoxides Alk-SNa, phenoxides Ar₂—ONa, or thiophenoxides Ar₂—SNa, with the product of general formula (D), by the action of sodium or sodium hydride on the alcohols or thiols, or by the action of sodium hydride or sodium hydroxide on the phenols or thiophenols in the solvent used for the reaction. The alkoxides, thioalkoxides, phenoxides, or thiophenoxides may be prepared beforehand or at the time of use. It has been discovered that the process may be performed by heating the mixture consisting of 1 mol of the product of general formula (D) with 1 mol of alcohol Alk-OH or of thiol Alk-SH or of phenol Ar₂—OH or of thiophenol Ar₂—SH, without solvent, by microwave irradiation. The process is preferably performed at a temperature of between 80 and 150° C.

Another subject of the present invention is thus a process for preparing compounds of formula (I) according to Scheme I. Generally, the product of general formula (D), as described above in which X represents a halogen atom and R, R₃ and Ar₁ have the meanings given above, is reacted with an alcohol Alk-OH, thiol Alk-SH, phenol Ar₂—OH, or a thiophenol Ar₂—SH without solvent, for example, by microwave radiation.

Such a process, may be performed at a temperature of between 80 and 150° C.

General method 2

According to a second method, the products of general formula (A), in which Ar is defined as above and R represents a group NR₁R₂ or OR₁ or SR₁, may also be prepared by nucleophilic displacement of a halogen atom. The method typically displaces a chlorine atom from a product of general formula (A), in which R represents a halogen atom, according to Scheme 2:

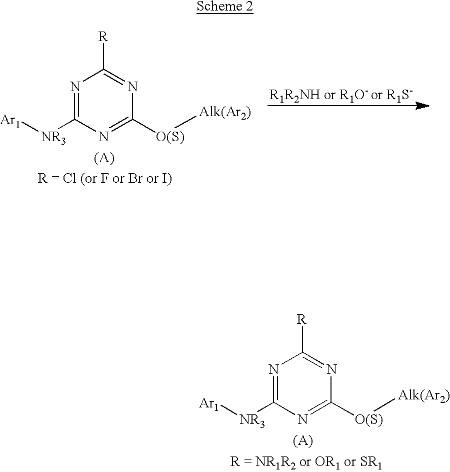

The process is generally performed by coupling 1 mol of product of general formula (A), in which R represents a halogen atom, e.g., a chlorine atom, with 1 mol of amine R₁R₂NH, alkoxide R₁O⁻, or of thioalkoxide R₁S. The reaction takes place in a medium which is generally inert under the reaction conditions. Among the inert solvents which may be mentioned are acetone, optionally aqueous acetone; or an optionally aqueous alcohol, such as ethanol; or a halogenated solvent, such as dichloromethane; or an ether, such as diethyl ether or dioxane; or a polar aprotic solvent, such as DMF, DMSO or NMP. When the entering group represents a group R₁R₂NH, the process is typically performed at a temperature of between 20° C. and reflux, in the presence of an organic base, such as triethylamine, or a mineral base, such as sodium hydroxide, sodium carbonate, or potassium carbonate. It is also possible not to use a base during this amination reaction, and to isolate a hydrochloride of the product of general formula (A), from which the base may then be released. When the entering group represents a group R₁O⁻ or R₁S⁻, the process is typically performed with an alkali metal, alkaline-earth metal alkoxide, thioalkoxide, such as a sodium, potassium, lithium, ammonium, cesium, or barium salt. Such a process is typically performed in a polar aprotic solvent such as DMF, DMSO, or NMP, at a temperature of between 50° C. and reflux.

General method 3

It is understood that the s-triazines, in general, may be obtained in the form of libraries by applying the methods described in Schemes 1 or 2 in parallel and/or combinatorial chemistry in liquid phase or in solid phase. It is understood that, when the process is performed in solid phase, any one of the reagents is bound beforehand to a solid support, chosen as a function of the chemical reaction taking place, and that the chemical reaction is followed by cleaving the reaction product from the solid support.

The present invention also relates to therapeutic compositions containing a compound according to the invention, in combination with a pharmaceutically acceptable carrier. Such a carrier is typically selected in accordance with the chosen mode of administration. The pharmaceutical composition may be in solid, liquid or liposomal form.

Suitable solid compositions include powders, gel capsules and tablets. Among the solid forms designed for oral administration, it is possible to provide solid forms that are protected against the acidic medium of the stomach. The carriers used in the solid forms may include mineral supports such as phosphates or carbonates, or organic supports such as lactose, celluloses, starch or polymers. The liquid forms may include solutions, suspensions or dispersions. Such forms may further contain a dispersive support, such as water, an organic solvent (ethanol, NMP or the like), mixtures of surfactants and solvents, or mixtures of complexing agents and solvents.

The administered dose of the compounds of the invention will be adapted by the practitioner as a function of the route of administration to a patient and the patient's condition.

The compounds of the present invention may be administered alone or mixed with other anticancer agents. Suitable anticancer agents include, but are not limited to:

alkylating agents such as cyclophosphamide, melphalan, ifosfamide, chlorambucil, busulfan, thiotepa, prednimustine, carmustine, lomustine, semustine, steptozotocin, decarbazine, temozolomide, procarbazine and hexamethylmelamine

platinum derivatives, such as cisplatin, carboplatin or oxaliplatin

antibiotics, such as bleomycin, mitomycin or dactinomycin,

antimicrotubule agents, such as vinblastine, vincristine, vindesine, vinorelbin and taxoids (paclitaxel and docetaxel)

anthracyclines, such as doxorubicin, daunorubicin, idarubicin, epirubicin, mitoxantrone or losoxantrone

topoisomerases of groups I and II, such as etoposide, teniposide, amsacrine, irinotecan, topotecan and tomudex,

fluoropyrimidines, such as 5-fluorouracil, UFT and floxuridine,

cytidine analogues, such as 5-azacytidine, cytarabine, gemcitabine, 6-mercaptomurine and 6-thioguanine

adenosine analogues, such as pentostatin, cytarabine and fludarabine phosphate

methotrexate and folinic acid

enzymes and various compounds, such as L-asparaginase, hydroxyurea, trans-retinoic acid, suramine, dexrazoxane, amifostine, herceptin and androgenic estrogenic hormones.

It is also possible to combine the compounds of the present invention with radiotherapy. These treatments may be administered simultaneously, separately or sequentially. The treatment will be adapted by a practitioner to the patient being treated.

The stabilizing activity of the above described compounds on G-quadruplexes may be determined by a method using a complex formed with fluorescein, as described below.

Oligonucleotides

All the oligonucleotides, modified or unmodified, were synthesized by Eurogentec S. A., Seraing, Belgium. The oligonucleotide FAM+DABCYL bears the catalogue reference OL-0371-0802. It has the sequence: GGGTTAGGGTTAGGGTTAGGG (SEQ ID NO:1) corresponding to 3.5 repetitions of the human telomeric motif (G-rich strand). Fluorescein is attached to the 5′ end and DABCYL to the 3′ end of the oligonucleotide, as described by Eurogentec. The concentration of the samples is checked by spectrophotometry, recording the absorbance spectrum between 220 and 700 nm and using the molar extinction coefficient provided by the supplier.

Buffers

All the experiments were performed in a 10 mM sodium cacodylate buffer, pH 7.6, containing 0.1 M of lithium chloride (or sodium chloride). The absence of fluorescent contamination in the buffer was confirmed beforehand. The fluorescent oligonucleotide is added to a final concentration of 0.2 μM.

Fluorescence Study

All the fluorescence measurements were carried out on a Spex Fluorolog DM1B machine, using an excitation line width of 1.8 nm and an emission line width of 4.5 nm. The samples are placed in a 0.2×1 cm quartz microcuvette. The temperature of the sample is controlled by an external water bath. The oligonucleotide alone was analyzed at 20, 30, 40, 50, 60, 70 and 80° C. The emission spectra are recorded using an excitation wavelength of 470 nm. The excitation spectra are recorded using either 515 nm or 588 nm as emission wavelength. The spectra are corrected for the response of the instrument by reference curves. A large (80-90%) extinction of the fluorescence of fluorescein at room temperature is observed, in accordance with an intramolecular fold of the oligonucleotide at 20° C. in the form of a G-quadruplex. Such folding induces a juxtaposition of its 5′ and 3′ ends, which are linked to fluorescein and DABCYL, respectively. This juxtaposition results in an already-described phenomenon of fluorescence extinction, which is used for “Molecular Beacons”.

Fluorescence Tm:

A stock solution of oligonucleotide at a strand concentration of 0.2 μM in a 0.1 M LiCl 10 mM cacodylate buffer (pH 7.6) is prepared beforehand, heated briefly to 90° C. cooled slowly to 20° C., and then distributed in 600 μl aliquots into fluorescence cuvettes. Three μl of water (for the control) or 3 μl of the product to be tested (stock at 200 μM, final concentration 1 μM) are then added and mixed. The samples are then left to incubate for at least 1 hour at 20° C. before each measurement. Using longer incubation times (up to 24 hours) has no influence on the results obtained.

Each experiment allows only one sample to be measured. This sample is first incubated at an initial temperature of 20° C., brought to 80° C. over 38 minutes, left at 80° C. for 5 minutes and then cooled to 20° C. for 62 minutes. During this time, the fluorescence is measured simultaneously at two emission wavelengths (515 nm and 588 nm) using 470 nm as excitation wavelength. A measurement is taken every 30 seconds. The temperature of the water bath is recorded in parallel, and the fluorescence profile as a function of the temperature is reconstituted from these values. The fluorescence profiles were normalized to between 20° C. and 80° C. The temperature for which the intensity of emission at 515 nm is the average of those at the high and low temperatures is referred to as Tm. Under these conditions, the Tm of the reference sample without the addition of product is 44° C. in a lithium chloride buffer. This temperature is brought to above 55° C. in a sodium chloride buffer. The addition of the G-quadruplex stabilizing compound induces an increase in the Tm. This increase is considered significant if it is greater than 3° C.

The anti-telomerase biological activity is determined by the following experimental protocol:

Preparation of the Extract Enriched in Human Telomerase Activity

The leukemia line HL60 is obtained from the ATCC (American Type Culture Collection, Rockville, USA). The cells are cultured in suspension in RPMI 1640 medium containing 2 MM L-glutamine, 200 U/ml penicillin, 200 μg/ml streptomycin, 50 μg/ml gentamycin and supplemented with 10% heat-inactivated fetal calf serum.

An aliquot of 10⁵ cells is centrifuged at 3000× g and the supernatant is discarded. The cell pellet is resuspended by pipetting several times successively in 200 μl of lysis buffer containing 0.5% CHAPS, 10 mM Tris-HCl pH 7.5, 1 mM MgCl₂, 1 mM EGTA, 5 mM β-mercaptoethanol, 0.1 mM PMSF and 10% glycerol and stored in ice for 30 minutes. The lysate is centrifuged at 16,000× g for 20 minutes at 4° C.; 160 μl of the supernatant are recovered. The assay of the proteins in the extract is carried out by the Bradford method. The extract is stored at −80° C.

Assay of the Telomerase Activity

The inhibition of the telomerase activity is determined by extension of the TS oligonucleotide ^{5′}AATCGTTCGAGCAGAGTT^{3′} (SEQ ID NO:2), in the presence of a cell extract enriched in telomerase activity and compounds which are added at various concentrations (10, 1, 0.1 and 0.01 μg/ml). The extension reaction is followed by a PCR amplification of the extension products using the oligonucleotides TS and CXext ^{5′}GTGCCCTTACCCTTACCCTTACCCTAA^{3′} (SEQ ID NO:3).

The reaction medium is prepared according to the following composition:

| | |
|---|---|
| Tris HCl pH 8.3 | 20 mM |
| MgCl2 | 1.5 mM |
| Tween 20 | 0.005% (P/V) |
| EGTA | 1 mM |
| dATP | 50 μM |
| dGTP | 50 μM |
| dCTP | 50 μM |
| dTTP | 50 μM |
| Oligonucleotide TS | 2 μg/ml |
| Oligonucleotide CXext | 2 μg/ml |
| Bovine serum albumin | 0.1 mg/ml |
| Taq DNA polymerase | 1 U/ml |
| alpha 32P dCTP (3000 Cimmol) | 0.5 μl |
| Telomerase extract | 200 ng in a |
| | volume of 10 μl |
| Product to be tested | in a volume |
| or solvent | of 5 μl |
| Double-distilled water qs | 50 μl |

The oligonucleotides are obtained from Eurogentec (Belgium) and are stored at −20° C. at a stock concentration of 1 mg/ml in distilled water.

The reaction samples are combined in 0.2 ml PCR tubes and one drop of liquid paraffin is placed in each of the reaction tubes before closing them.

The reaction samples are then incubated in a PCR machine, such as a Cetus 4800, under the following temperature conditions:

15 minutes at 30° C.,

1 minute at 90° C.,

followed by 30 cycles of:

30 seconds at 94° C.,

30 seconds at 50° C., and

1 minute 30 seconds at 72° C.,

followed by a final cycle of 2 minutes at 72° C.

For each of the samples, a 10 μl aliquot is pipetted under the layer of oil and mixed with 5 μl of a deposit buffer containing

| TBE | 3X |
|---|---|
| Glycerol | 32% (W/V) |
| Bromophenol blue | 0.03% |
| Xylene cyanol | 0.03% |

The samples are then analyzed by electrophoresis on 12% acrylamide gel in a TBE 1× buffer for 1 hour under a tension of 200 volts, using a Novex electrophoresis system.

The acrylamide gels are then dried on a sheet of Whatmann 3MM paper at 80° C. for 1 hour.

The analysis and quantification of the reaction products are carried out using an InstantImager machine (Pacard).

For each concentration of compound tested, the results are expressed as a percentage of inhibition of the reaction and calculated from the untreated enzymatic control and from the enzyme-free sample (blank) according to the following formula:

(Compound value−blank value/Enzymatic control value−blank value)×100.

The concentration of compound which induces a 50% inhibition of the telomerase reaction (IC50) is determined using a semilogarithmic graphical representation of the inhibition values obtained as a function of each of the concentrations of compound tested.

A compound is considered to be active as an anti-telomerase agent when the amount inhibiting the telomerase reaction by 50% is less than 5 μM.

The Cytotoxic Biological Activity on Human Tumor Lines is Determined According to the Following Experimental Protocol:

The human cell lines KB and A549 are obtained from the ATCC (American Type Culture Collection, Rockville, USA). The A549 cells are cultured as a layer in a culture flask in RPMI 1640 medium, L-glutamine to 2 mM, 200 U/ml penicillin, 200 μg/ml streptomycin and supplemented with 10% heat-inactivated fetal calf serum. The KB cells are cultured as a layer in a culture flask in Dulbelco's medium containing L-glutamine to 2 mM, 200 U/ml penicillin, 200 μg/ml streptomycin and supplemented with 10% heat-inactivated fetal calf serum.

The cells in exponential growth phase are trypsinized, washed in PBS 1×, and inoculated in 96-well microplates (Costar) at a concentration of 4×10⁴ cells/ml for A549 and 1.5×10⁴ cells/ml (0.2 ml/well) for KB cells. The cells are then incubated for 96 hours in the presence of variable concentrations of test compounds (10, 1, 0.1 and 0.01 μg/ml, each point being determined in quadruplicate). Sixteen hours before the end of the incubation, neutral red is added to a final concentration of 0.02% in each well. At the end of the incubation, the cells are washed with PBS 1× and lysed with 1% sodium lauryl sulfate. The cellular incorporation of the dye, which reflects the cell growth, is evaluated by spectrophotometry at a wavelength of 540 nm for each sample using a Dynatech MR5000 reading machine.

For each concentration of compound tested, the results are expressed as a percentage of inhibition of cell growth, calculated from the untreated control and the cell-free culture medium (blank) according to the following formula:

(Compound value−blank value/Cell control value−blank value)×100.

The concentration of compound which induces a 50% inhibition of growth (IC50) is determined using a semilogarithmic graphical representation of the inhibition values obtained as a function of each of the concentrations of compound tested.

A compound is considered to be active as a cytotoxic agent if the concentration inhibiting 50% of the growth of the tumor cells tested is less than 10 μM.

The non-limiting examples which follow are given to illustrate the invention.

### EXAMPLE 1

### Synthesis of N6-[6-(2-dimethylaminoethoxy)-4-methylsulfanyl-[1,3,5]triazin-2-yl]-2-methylquinoline-4,6-diamine

Preparation of N6-(6-chloro-4-methylsulfanyl-[1,3,5]triazin-2-yl)-2-methylquinoline-4,6-diamine

4.4 g (25 mmol) of 2-methylquinoline-4,6-diamine, (which may be prepared according to J. Med. Chem., 35:252, 1992), and 2.8 g (25 mmol) of sodium carbonate were successively added to a solution of 5 g (25 mmol) of 2,6-dichloro-6-methylsulfanyl-[1,3,5]triazine, (which may be prepared according to J. Amer. Chem. Soc., 67:662, 1945), in 400 ml of tetrahydrofuran, in a 1 liter 3-necked flask.

The reaction mixture was refluxed for 16 hours. After evaporating off the tetrahydrofuran, the residue was taken up in 400 ml of a mixture of water and dichloromethane (50/50 by volume). The organic phase was separated out after settling had taken place, dried over sodium sulfate and concentrated to dryness under reduced pressure. 7.5 g (88%) of N6-(6-chloro-4-methylsulfanyltriazin-2-yl)-2-methylquinoline-4,6-diamine were thus obtained in the form of a pale yellow solid, the characteristics of which are as follows:

Melting point=294° C.

¹H NMR spectrum (300 MHz, d₆-(CD₃)₂SO, δ in ppm): 2.43 (s: 3H); 2.52 (s: 3H); 6.47 (s: 1H); 6.61 (multiplet: 2H); 7.62 (broad d, J=9 Hz: 1H); 7.69 (d, J=9 Hz: 1H); 8.32 (multiplet: 1H); 10.80 (multiplet: 1H).

Preparation of N6-[6-(2-dimethylaminoethyloxy)-4-methylsulfanyl-[1,3,5]triazin-2-yl]-2-methylquinoline-4,6-diamine (Example 1)

25 mg (0.075 mmol) of N6-(6-amino-4-methylsulfanyl-[1,3,5]triazin-2-yl)-2-methylquinoline-4,6-diamine and 27 μl (0.27 mmol) of 2-dimethylaminoethanol were introduced into a Synthewave 402 Prolabo 12 ml quartz microwave reactor. The mixture was heated for 2 hours by microwave irradiation at a temperature of about 80° C. After cooling, the contents of the tube were taken up in methanol and evaporated under reduced pressure. The residue was crystallized from toluene. 26 mg (90% yield) of N6-[6-(2-dimethylaminoethyloxy)-4-methylsulfanyl-[1,3,5]triazin-2-yl]-2-methylquinoline-4,6-diamine were thus obtained in the form of an apricot-yellow powder, the characteristic of which is as follows:

Mass spectrum (EI/DCI)=385 (MH⁺).

### EXAMPLE 2

### Synthesis of N6-[6-(3-dimethylaminopropoxy)-4-methylsulfanyl-[1,3,5]triazin-2-yl]-2-methylquinoline-4,6-diamine

25 mg (0.075 mmol) of N6-(6-amino-4-methylsulfanyl-[1,3,5]triazin-2-yl)-2-methylquinoline-4,6-diamine, prepared as in Example 1, and 35 μl (0.3 mmol) of 3-dimethylaminopropanol were introduced into a Synthewave 402 Prolabo 12 ml quartz microwave reactor. The mixture is heated for 5 minutes under microwave irradiation at a temperature of about 125° C. After cooling, the contents of the tube were taken up in methanol and evaporated under reduced pressure. The residue was crystallized from toluene. 25 mg (83% yield) of N6-[6-(2-dimethylaminoethyloxy)-4-methylsulfanyl-[1,3,5]triazin-2-yl]-2-methylquinoline-4,6-diamine were thus obtained in the form of an apricot-yellow powder, the characteristic of which is as follows:

Mass spectrum (EI/DCI)=399 (MH⁺).

### EXAMPLE 3

### Synthesis of N6-[6-(1-methylpiperid-4-yloxy)-4-methylsulfanyl-[1,3,5]triazin-2-yl]-2-methylquinoline-4,6-diamine

25 mg (0.075 mmol) of N6-(6-amino-4-methylsulfanyl-[1,3,5]triazin-2-yl)-2-methylquinoline-4,6-diamine (prepared as in Example 1), 35 mg (0.3 mmol) of 4-hydroxy-1-methylpiperidine, and 0.5 g of alumina were introduced into a Synthewave 402 Prolabo 12 ml quartz microwave reactor. The mixture was heated for 10 minutes under microwave irradiation at a temperature of about 148° C. After cooling, the contents of the tube were taken up in methanol and evaporated under reduced pressure. The residue was crystallized from toluene. 22 mg (71% yield) of N6-[6-(1-methylpiperid-4-yloxy)-4-methylsulfanyl-[1,3,5]triazin-2-yl]-2-methylquinoline-4,6-diamine were obtained in the form of a pale yellow powder, the characteristic of which is as follows: Mass spectrum (EI/DCI)=411 (MH⁺).

### EXAMPLE 4

### Synthesis of N6-[6-(2-dimethylaminoethylsulfanyl)-4-methylsulfanyl-[1,3,5]triazin-2-yl]-2-methylquinoline-4,6-diamine

2.5 ml of dioxane, 50 μl (0.31 mmol) of 2-methylaminoethanethiol hydrochloride, 50 μl of 10 N sodium hydroxide solution and 25 mg (0.075 mmol) of N6-(6-amino-4-methylsulfanyl-[1,3,5]triazin-2-yl)-2-methylquinoline-4,6-diamine (prepared as in Example 1), were successively introduced into a 10 ml three-necked flask. The mixture was refluxed for 16 hours. After cooling, the precipitate formed was spin-filtered and crystallized from diethyl ether. 17 mg (57% yield) of N6-[6-(2-dimethylaminoethylsulfanyl)-4-methylsulfanyl-[1,3,5]triazin-2-yl]-2-methylquinoline-4,6-diamine were thus obtained in the form of a beige-colored powder, the characteristic of which is as follows:

Mass spectrum (EI/DCI)=401 (MH⁺).

Table of biological results

| | TRAP | G-4 | Cytotoxicity |
|---|---|---|---|
| | Telomerase | Delta Tm | A549 |
| Example | IC50 μM | ° C. | IC50 μM |
| 1 | 3.3 | 2.6 | |
| 2 | 2 | 6.2 | |
| 3 | 0.87 | 4.5 | 6.0 |
| 4 | 0.66 | 4.1 | |

We claim:

1. A compound that binds a G-quadruplex structure of DNA or RNA, comprising the following general formula:
nitrogenous aromatic ring —NR₃— distributor —O or S— non-aromatic hydrocarbon-based chain or nitrogenous aromatic ring
in which
1) the nitrogenous aromatic ring represents:
a) a quinoline optionally substituted with at least
i) one group N(Ra)(Rb) in which Ra and Rb are identical or different and represent hydrogen or a C1-C4 alkyl radical, or
ii) a group ORa in which Ra is defined as above,
b) a quinoline containing a nitrogen atom in quaternary form, or
c) a benzamidine, or
d) a pyridine;
2) R₃ represents hydrogen or a C1-C4 alkyl radical;
3) the distributor represents:
a) a triazine group, unsubstituted or substituted with an alkyl radical containing 1 to 4 carbon atoms, a thio radical, an oxy radical, or an amino radical, wherein the thio, oxy, or amino radicals are unsubsituted or substituted with (i) one or more short-chain alkyl chains containing 1 to 4 carbon atoms or (ii) a halogen atom, or
b) a carbonyl group, or
c) a C(═NH)—NH—C(═NH) group, or
d) an alkyldiyl group containing 3 to 7 carbon atoms, or
e) a diazine group, unsubstituted or substituted with an alkyl radical containing 1 to 4 carbon atoms, a thio radical, an oxy radical, or an amino radical, wherein the thio, oxy, or amino radicals are unsubsituted or substituted with (i) one or more short-chain alkyl chains containing 1 to 4 carbon atoms or (ii) a halogen atom;
or a salt thereof.

2. The compound of claim 1, wherein the compound binds to a telomere.

3. The compound of claim 1, wherein the distributor is chosen from triazine and diazine groups.

4. The compound of claim 1, wherein the diazine group is a pyrimidine or quinazoline group.

5. The compound of claim 1, wherein the non-aromatic hydrocarbon-based chain is chosen from linear or branched alkyl (C1-C4), linear or branched alkenyl (C2-C4), cycloalkyl (C3-C18), cycloalkenyl (C3-C18), heterocycloalkyl (C3-C18), and heterocycloalkyl (C3-C18) including the nitrogen atom of an amino, alkylamino, arylamino, dialkyl amino, diarylamino, or combination thereof.

6. The compound of claim 1, wherein the non-aromatic hydrocarbon-based chain is unsubstituted or substituted with one or more groups chosen from halogen, hydroxyl, aryl, heteroaryl, alkyloxy, aryloxy, thio, alkylthio, arylthio, amino, alkylamino, arylamino, dialkylamino, diarylamino, amidino, guanidino, alkylcarbonylamino, arylcarbonylamino, carboxyl, alkyloxycarbonyl, aryloxycarbonyl, aminocarbonyl, alkylaminocarbonyl, arylaminocarbonyl, dialkylaminocarbonyl, alkylcarbonyl, arylcarbonyl, cyano, trifluoromethyl, and combinations thereof.

7. The compound of claim 6, wherein the alkyl groups substituted on the non-aromatic hydrocarbon-based chain contain 1 to 4 carbon atoms and the aryl groups substituted on the non-aromatic hydrocarbon-based chain contain 5 to 18 carbon atoms.

8. The compound of claim 5, wherein the non-aromatic hydrocarbon-based chain is an alkyl containing 2 or 3 carbon atoms, a heterocycloalkyl containing 5 to 7 carbon atoms, or a cycloalkyl containing 5 to 7 carbon atoms.

9. The compound of claim 1, comprising formula (I) below: 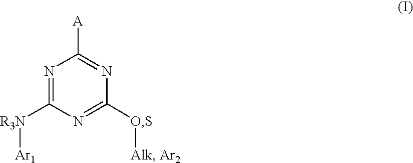
in which:
1) the triazine ring bears an oxygen or sulfur atom which is itself linked to Alk or Ar₂,
2) A represents
a) an amino group of formula NR₁R₂ in which R₁ and R₂ are identical or different and represent hydrogen or a straight or branched alkyl group containing 1 to 4 carbon atoms, or
b) a group OR₁ or SR₁ in which R₁ has the same meaning as above, or
c) an alkyl group containing 1 to 4 carbon atoms or a trifluoromethyl group, or
d) a hydrogen atom, or
e) a halogen atom chosen from fluorine, chlorine, bromine and iodine;
3) R₃ represents hydrogen or a C1-C4 alkyl group;
4) Ar₁ and Ar₂, which are identical or different, represent:
a) a quinoline optionally substituted with at least
i) one group N(Ra)(Rb) in which Ra and Rb are identical or different and represent hydrogen or a C1-C4 alkyl radical, or
ii) a group ORa in which Ra is defined as above,
b) a quinoline containing a nitrogen atom in quaternary form, or
c) a benzamidine, or
d) a pyridine attached in position −4 or fused with an aryl or heteroaryl group that is unsubstituted or substituted with a C1-C4 alkyl group;
5) Alk represents an unsubstituted or substituted non-aromatic hydrocarbon-based chain chosen from linear or branched alkyl (C1-C4), linear or branched alkenyl (C2-C4), cycloalkyl (C3-C18), cycloalkenyl (C3-C18), heterocycloalkyl (C3-C18), and heterocycloalkyl (C3-C18) including the nitrogen atom of an amino, alkylamino, arylamino, dialkylamino, diarylamino, or combination thereof;
or a salt thereof.

10. The compound of claim 9, wherein the non-aromatic hydrocarbon-based chain Alk is unsubstituted or substituted with one or more groups chosen from halogen, hydroxyl, aryl, heteroaryl, alkyloxy, aryloxy, thio, alkylthio, arylthio, amino, alkylamino, arylamino, dialkylamino, diarylamino, amidino, guanidino, alkylcarbonylamino, arylcarbonylamino, carboxyl, alkyloxycarbonyl, aryloxycarbonyl, aminocarbonyl, alkylaminocarbonyl, arylaminocarbonyl, dialkylaminocarbonyl, alkylcarbonyl, arylcarbonyl, cyano, trifluoromethyl, and combinations thereof.

11. The compound of claim 9, wherein Ar₁ represents 4-amino- or 4-methylamino- or 4-dimethylamino-quinolyl or quinolinium in which the quinolinium ring is unsubstituted or substituted with a methyl group.

12. The compound of claim 9, wherein A represents a methylthio, amino, alkylamino or dialkylamino radical, and wherein the alkyl groups in the radicals contain 1 to 4 carbon atoms.

13. The compound of claim 9, wherein A represents a methylthio group.

14. The compound of claim 9, wherein Alk represents:
1) a linear or branched alkyl unit containing 2 or 3 carbon atoms that is substituted with an amino, alkylamino, arylamino, dialkylamino, diarylamino, or combination thereof,
2) an alkenyl unit containing 2 or 3 carbon atoms that is substituted with an amino, alkylamino, arylamino, dialkylamino, diarylamino, or combination thereof,
3) a cycloalkyl unit containing from 4 to 7 carbon atoms, or
4) a heterocycloalkyl (C₄-C₇) unit including the nitrogen atom of an amino, alkylamino, arylamino, dialkylamino, diarylamino, or combination thereof.

15. The compound of claim 9, characterized in that Alk represents a 2-(dialkylamino)ethyl, 3-(dialkylamino)propyl, 2-(N-alkyl-N-arylamino)ethyl, 3-(N-alkyl-N-arylamino)propyl, N-alkylpiperidyl, or N-arylpiperidyl chain in which the alkyl groups contain 1 to 4 carbon atoms and the aryl groups contain 5 to 18 carbon atoms.

16. The compound of claim 1, wherein the compound inhibits telomerase activity.

17. The compound of claim 1, wherein the compound has anticancer activity.

18. A compound comprising the following general formula:
nitrogenous aromatic ring —NR₃— distributor —O— or —S— non-aromatic hydrocarbon-based chain or nitrogenous aromatic ring
in which
1) the nitrogenous aromatic ring represents:
a) a quinoline optionally substituted with at least
i) one group N(Ra)(Rb) in which Ra and Rb are identical or different and represent hydrogen or a C₁-C₄ alkyl radical, or
ii) a group ORa in which Ra is defined as above,
b) a quinoline containing a nitrogen atom in quaternary form, or
c) a benzamidine, or
d) a pyridine;
2) R₃ represents hydrogen or a C1-C4 alkyl radical
3) the distributor represents:
a) a triazine group that is unsubstituted or substituted with an alkyl radical containing 1 to 4 carbon atoms, a thio radical, an oxy radical, or an amino radical, wherein the thio, oxy, or amino radicals are unsubstituted or substituted with (i) one or more short-chain alkyl chains containing 1 to 4 carbon atoms or (ii) a halogen atom, or
b) a diazine group that is unsubstituted or substituted with an alkyl radical containing 1 to 4 carbon atoms, a thio radical, an oxy radical, or an amino radical, wherein the thio, oxy, or amino radicals are unsubstituted or substituted with (i) one or more short-chain alkyl chains containing 1 to 4 carbon atoms or (ii) a halogen atom;
4) the non-aromatic hydrocarbon-based chain is chosen from linear or branched alkyl (C1-C4), linear or branched alkenyl (C2-C4), cycloalkyl (C3-C18), cycloalkenyl (C3-C18), heterocycloalkyl (C3-C18), and heterocycloalkyl (C3-C18) chains including the nitrogen atom of an amino, alkylamino, arylamino, dialkylamino, diarylamino, or combination thereof;
or a salt thereof.

19. Compounds corresponding to formula (I) below: 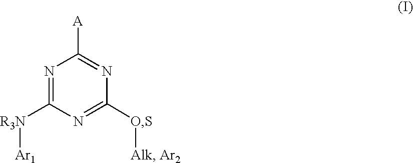
in which:
1) A represents
a) an amino group of formula NR₁R₂ in which R₁ and R₂, are identical or different and represent a straight or branched alkyl group containing 1 to 4 carbon atoms, or
b) a group OR₁ or SR₁ in which R₁ represents hydrogen or has the same meaning as above, or
c) an alkyl group containing 1 to 4 carbon atoms or a trifluoromethyl group, or
d) a hydrogen atom, or
e) a halogen atom chosen from fluorine, chlorine, bromine and iodine;
2) R₃ represents a hydrogen atom or a C1-C4 alkyl group;
3) Ar₁ and Ar₂, which are identical or different, represent:
a) a quinoline optionally substituted with at least
i) one group N(Ra)(Rb) in which Ra and Rb are identical or different and represent hydrogen or a C1-C4 alkyl radical, or
ii) a group ORa in which Ra is defined as above,
b) a quinoline containing a nitrogen atom in quaternary form, or
c) a benzamidine, or
d) a pyridine attached in position −4 or fused with an aryl or heteroaryl group that is unsubstituted or substituted with a C1-C4 alkyl group
4) Alk represents an unsubstituted or substituted non-aromatic hydrocarbon-based chain chosen from linear or branched alkyl (C1-C4), alkenyl (C2-C4), cycloalkyl (C3-C18), cycloalkenyl (C3-C18), heterocycloalkyl (C3-C18), and heterocycloakyl (C3-C18) including the nitrogen atom of an amino, alkylamino, arylamino, dialkylamino, diarylamino, or combination thereof;
or a salt thereof.

20. The compounds of claim 18, wherein the non-aromatic hydrocarbon-based chain Alk is unsubstituted or substituted with one or more groups chosen from halogen, hydroxyl, aryl, heteroaryl, alkyloxy, aryloxy, thio, alkylthio, arylthio, amino, alkylamino, arylamino, dialkylamino, diarylamino, amidino, guanidino, alkylcarbonylamino, arylcarbonylamino, carboxyl, alkyloxycarbonyl, aryloxycarbonyl, aminocarbonyl, alkylaminocarbonyl, arylaminocarbonyl, dialkylaminocarbonyl, alkylcarbonyl, arylcarbonyl, cyano, trifluoromethyl, and combinations thereof.

21. The compound of claim 19, wherein Ar₁ and Ar₂ are identical or different and represent 4-amino- or 4-methylamino- or 4-dimethylamino-quinolyl or quinolinium, wherein the quinolinium ring is unsubstituted or substituted with a methyl group.

22. The compound of claim 19, wherein A represents a methylthio, amino, alkylamino or dialkylamino radical, wherein the alkyl groups in the radicals contain 1 to 4 carbon atoms.

23. The compound of claim 19, wherein R₁ and R₂ represent hydrogen.

24. The compound of claim 22, wherein A represents a methylthio group.

25. The compound of claim 19, wherein Alk represents
i) a linear or branched alkyl unit containing 2 or 3 carbon atoms, substituted with an amino, alkylamino, arylamino, dialkylamino, diarylamino, or combination thereof, or
ii) an alkenyl unit containing 2 or 3 carbon atoms, substituted with an amino, alkylamino, arylamino, dialkylamino, diarylamino, or combination thereof, or
iii) a heterocycloalkyl or cycloalkyl unit containing from 4 to 7 carbon atoms.

26. The compound of claim 19, wherein Alk represents a 2-(dialkylamino)ethyl, 3-(dialkylamino)propyl, 2-(N-alkyl-N-arylamino)ethyl, 3-(N-alkyl-N-arylamino)propyl, N-alkylpiperidyl or N-arylpiperidyl chain in which the alkyl groups contain 1 to 4 carbon atoms and the aryl groups contain 5 to 18 carbon atoms.

27. The compound of claim 18, selected from among:
N6-[6-(2-dimethylaminoethoxy)-4-methylsulfanyl-[1,3,5]triazin-2-yl]-2-methylquinoline-4,6-diamine,
N6-[6-(3-dimethylaminopropoxy)-4-methylsulfanyl-[1,3,5]triazin-2-yl]-2-methylquinoline-4,6-diamine,
N6-[6-(1-methylpiperidin-4-yloxy)-4-methylsulfanyl-[1,3,5]triazin-2-yl]-2-methylquinoline-4,6-diamine, and
N6-[6-(2-dimethylaminoethylsulfanyl)-4-methylsulfanyl-[1,3,5]triazin-2-yl]-2-methylquinoline-4,6-diamine.

28. A pharmaceutical product for human use comprising one or more compounds of claim 1.

29. A therapeutic composition comprising one or more compounds of claim 1 and one or more anticancer agents.

30. The composition according to claim 29, wherein the one or more anticancer agents are chosen from alkylating agents, platinum derivatives, antibiotics, antimicrotubule agents, anthracyclines, topoisomerases of groups I and II, fluoropyrimidines, cytidine analogues, adenosine analogues, L-asparaginase, hydroxyurea, trans-retinoic acid, suramine, irinotecan, topotecan, dexrazoxane, amifostine, herceptin, androgenic hormones, and estrogenic hormones.

31. A therapeutic combination comprising one or more compounds of claim 1 and radiation.

32. A method of using the composition of claim 29, wherein the individual compounds are administered to a patient simultaneously, separately or sequentially.

33. A process for preparing the compounds of claim 9, wherein a compound comprising general formula (D) 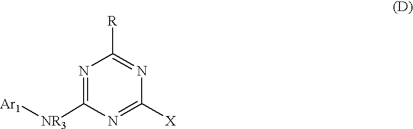
in which R represents the values of A as defined in claim 9,
X represents a halogen atom, and
R₃ and Ar₁ have the meanings given in claim 9, is reacted with an alcohol Alk-OH, a thiol Alk-SH, a phenol
Ar₂—OH, or a thiophenol Ar₂—SH, and
wherein the reaction is performed, without adding a solvent, by microwave irradiation.

34. The process of claim 33, wherein the reaction is performed at a temperature of between 80 and 150° C.

35. The compound of claim 19, selected from among:
N6-[6-(2-dimethylaminoethoxy)-4-methylsulfanyl-[1,3,5]triazin-2-yl]-2-methylquinoline-4,6-diamine,
N6-[6-(3-dimethylaminopropoxy)-4-methylsulfanyl-[1,3,5]triazin-2-yl]-2-methylquinoline-4,6-diamine,
N6-[6-(1-methylpiperidin-4-yloxy)-4-methylsulfanyl-[1,3,5]triazin-2-yl]-2-methylquinoline-4,6-diamine, and
N6-[6-(2-dimethylaminoethylsulfanyl)-4-methylsulfanyl-[1,3,5]triazin-2-yl]-2-methylquinoline-4,6-diamine.
